(19) United States
(12) Patent Application Publication
(10) Pub. No.: US 2019/0008390 A1
(43) Pub. Date: Jan. 10, 2019

(54) Programmable Swept Frequency Light Source

(71) Applicant: Carestream Dental Technology Topco Limited, London (GB)

(72) Inventors: Victor WONG, Pittsford, NY (US); Chuanmao FAN, Rochester, NY (US)

(21) Appl. No.: 16/067,599

(22) PCT Filed: Dec. 30, 2015
(86) PCT No.: PCT/US2015/068028

§ 371 Date: Jun. 30, 2018

(51) Int. Cl. A61B 5/00; H01S 3/00; H01S 3/067; G02B 26/08; G01B 9/02

(52) U.S. Cl. CPC A61B 5/0066; A61B 5/0088; H01S 3/0078; H01S 3/0071; A61B 2562/0233; G02B 26/0833; G01B 9/02004; G01B 9/02091; H01S 3/06791

(57) ABSTRACT

A programmable light source has a broadband light emitter disposed to direct light to an optical filter. The optical filter has a collimator lens in the path of the directed light from the emitter, a dispersion optic in the path of incident light from the collimator lens and angularly disposed to form a spectrally dispersed output beam from the incident beam, a focusing lens in the path of the spectrally dispersed output beam, and a spatial light modulator in the focal region of the focusing lens, the spatial light modulator disposed to reflect sequential spectral portions of the spectrally dispersed output beam back toward the focusing lens.

### FIELD OF THE INVENTION

The disclosure relates generally to spectrally variable light sources and more particularly to a light source suitable for swept source optical coherence tomography imaging.

### BACKGROUND OF THE INVENTION

Optical coherence tomography (OCT) is a non-invasive imaging technique that employs interferometric principles to obtain high resolution, cross-sectional tomographic images that characterize the depth structure of a sample. Particularly suitable for in vivo imaging of human tissue, OCT has shown its usefulness in a range of biomedical research and medical imaging applications, such as in ophthalmology, dermatology, oncology, and other fields, as well as in ear-nose-throat (ENT) and dental imaging.

OCT has been described as a type of “optical ultrasound”, imaging reflected energy from within living tissue to obtain cross-sectional data. In an OCT imaging system, light from a wide-bandwidth source, such as a super luminescent diode (SLD) or other light source, is directed along two different optical paths: a reference arm of known length and a sample arm that illuminates the tissue or other subject under study. Reflected and back-scattered light from the reference and sample arms is then recombined in the OCT apparatus and interference effects are used to determine characteristics of the surface and near-surface underlying structure of the sample. Interference data can be acquired by rapidly scanning the sample illumination across the sample. At each of several thousand points, OCT apparatus obtains an interference profile which can be used to reconstruct an A-scan with an axial depth into the material that is a factor of light source coherence. For most tissue imaging applications, OCT uses broadband illumination sources and can provide image content at depths of a few millimeters (mm).

Initial OCT apparatus employed a time-domain (TD-OCT) architecture in which depth scanning is achieved by rapidly changing the length of the reference arm using some type of mechanical mechanism, such as a piezoelectric actuator, for example. TD-OCT methods use point-by-point scanning, requiring that the illumination probe be moved or scanned from one position to the next during the imaging session. More recent OCT apparatus use a Fourier-domain architecture (FD-OCT) that discriminates reflections from different depths according to the optical frequencies of the signals they generate. FD-OCT methods simplify or eliminate axial scan requirements by collecting information from multiple depths simultaneously and offer improved acquisition rate and signal-to-noise ratio (SNR). There are two implementations of Fourier-domain OCT: spectral domain OCT (SD-OCT) and swept-source OCT (SS-OCT).

SD-OCT imaging can be accomplished by illuminating the sample with a broadband source and dispersing the reflected and scattered light with a spectrometer onto an array detector, such as a CCD (charge-coupled device) detector, for example. SS-OCT imaging illuminates the sample with a rapid wavelength-tuned laser and collects light reflected during a wavelength sweep using only a single photodetector or balanced photodetector. With both SD-OCT and SS-OCT, a profile of scattered light reflected from different depths is obtained by operating on the recorded interference signals using Fourier transforms, such as Fast-Fourier transforms (FFT), well known to those skilled in the signal analysis arts.

Because of their potential to achieve higher performance at lower cost, FD-OCT systems based on swept-frequency laser sources have attracted significant attention for medical applications that require subsurface imaging in highly scattering tissues.

One of the challenges to SS-OCT is providing a suitable light source that can generate the needed sequence of wavelengths in rapid succession. To meet this need, swept-source OCT systems conventionally employ a high-speed wavelength sweeping laser that is equipped with an intracavity monochrometer or uses some type of external cavity narrowband wavelength scanning filter for tuning laser output. Examples of external devices that have been used for this purpose include a tunable Fabry-Perot filter whose cavity length is adjusted to provide a linear change of longitudinal mode, and a polygon scanner filter that selectively reflects dispersive wavelength light. Fourier domain mode locking is a recently reported technique that has been used to generate a sweeping frequency.

References for providing a tunable laser include the following:
S. R. Chinn, E. A. Swanson, and J. G. Fujimoto, “Optical coherence tomography using a frequency-tunable optical source,” Opt. Lett. 22, 340-342 (1997).
B. Golubovic, B. E. Bouma, G. J. Tearney, and J. G. Fujimoto, “Optical frequency-domain reflectometry using rapid wavelength tuning of a Cr4+:forsterite laser,” Opt. Lett. 22, 1704-1706 (1997).
S. H. Yun, C. Boudoux, G. J. Tearney, and B. E. Bouma, “High-speed wavelength-swept semiconductor laser with a polygon-scanner-based wavelength filter,” Opt. Lett. 28, 1981-1983 (2003).
Woojin Shin, Boan-Ahn Yu, Yeung Lak Lee, Tae Jun Yu, Tae Joong Eom, Young-Chul Noh, Jongmin Lee, and Do-Kyeong Ko, “Tunable Q-switched erbium-doped fiber laser based on digital micromirror array,” Opt. Express 14, 5356-5364 (2006),
Xiao Chen, Bin-bin Yan, Fei-jun Song, Yi-quan Wang, Feng Xiao, and Kamal Alameh, “Diffraction of digital micro-mirror device gratings and its effect on properties of tunable fiber lasers,” Appl. Opt. 51, 7214-7220 (2012).

The conventional approaches for providing swept source illumination enable SS-OCT imaging but can be costly and complex and are limited to fixed wavelength sequences. Detection of moving subjects, for example, is limited. Thus, there is a need for more flexible illumination solutions that support SS-OCT and other applications that benefit from a changing spectral pattern.

### SUMMARY OF THE INVENTION

It is an object of the present disclosure to advance the art of illumination using a pattern of variable wavelengths. An embodiment of the present disclosure obtains a programmable sequence of light wavelengths from a broadband light source that can be particularly suitable for a range of spectral imaging applications including use in portable optical coherence tomography apparatus.

These objects are given only by way of illustrative example, and such objects may be exemplary of one or more embodiments of the invention. Other desirable objectives and advantages inherently achieved by the disclosed methods may occur or become apparent to those skilled in the art. The invention is defined by the appended claims.

According to an aspect of the present disclosure, there is provided a programmable light source comprising: a) a broadband light emitter disposed to direct light to an optical filter; b) the optical filter having: (i) a collimator lens in the path of the directed light from the emitter; (ii) a dispersion optic in the path of incident light from the collimator lens and angularly disposed to form a spectrally dispersed output beam from the incident beam; (iii) a focusing lens in the path of the spectrally dispersed output beam; (iv) a spatial light modulator in the focal region of the focusing lens, the spatial light modulator disposed to reflect sequential spectral portions of the spectrally dispersed output beam back toward the focusing lens.

### BRIEF DESCRIPTION OF THE DRAWINGS

The foregoing and other objects, features, and advantages of the invention will be apparent from the following more particular description of the embodiments of the invention, as illustrated in the accompanying drawings.

The elements of the drawings are not necessarily to scale relative to each other. Some exaggeration may be necessary in order to emphasize basic structural relationships or principles of operation. Some conventional components that would be needed for implementation of the described embodiments, such as support components used for providing power, for packaging, and for mounting and protecting system optics, for example, are not shown in the drawings in order to simplify description.

FIG. 1 is a schematic diagram that shows a programmable filter according to an embodiment of the present disclosure.

FIG. 2A is a simplified schematic diagram that shows how the programmable filter provides light of a selected wavelength band.

FIG. 2B is an enlarged view of a portion of the micro-mirror array of the programmable filter.

FIG. 3 is a plan view that shows the arrangement of micro-mirrors in the array.

FIG. 4 is a schematic diagram that shows a programmable filter using a prism as its dispersion optic, according to an alternate embodiment of the present disclosure.

FIG. 5 is a schematic diagram showing a programmable filter that performs wavelength-to-wavenumber transformation, according to an alternate embodiment of the present disclosure.

FIG. 6A is a schematic diagram showing a swept-source OCT (SS-OCT) apparatus using a programmable filter according to an embodiment of the present disclosure that uses a Mach-Zehnder interferometer.

FIG. 6B is a schematic diagram showing a swept-source OCT (SS-OCT) apparatus using a programmable filter according to an embodiment of the present disclosure that uses a Michelson interferometer.

FIG. 7 is a schematic diagram that shows a tunable laser using a programmable filter according to an embodiment of the present disclosure.

FIG. 8 is a schematic diagram that shows use of a programmable filter for selecting a wavelength band from a broadband light source.

FIG. 9 shows galvo mirrors used to provide a 2-D scan as part of the OCT imaging system probe.

FIG. 10A shows a schematic representation of scanning operation for obtaining a B-scan.

FIG. 10B shows an OCT scanning pattern for C-scan acquisition.

FIG. 11 is a schematic diagram that shows components of an intraoral OCT imaging system.

### DETAILED DESCRIPTION OF THE INVENTION

The following is a detailed description of the preferred embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

As used herein, the term “energizable” relates to a device or set of components that perform an indicated function upon receiving power and, optionally, upon receiving an enabling signal.

In the context of the present disclosure, the term “optics” is used generally to refer to lenses and other refractive, diffractive, and reflective components or apertures used for shaping and orienting a light beam. An individual component of this type is termed an optic.

In the context of the present disclosure, the terms “viewer”, “operator”, and “user” are considered to be equivalent and refer to the viewing practitioner, technician, or other person who may operate a camera or scanner and may also view and manipulate an image, such as a dental image, on a display monitor. An “operator instruction” or “viewer instruction” is obtained from explicit commands entered by the viewer, such as by clicking a button on the camera or scanner or by using a computer mouse or by touch screen or keyboard entry.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the terms “camera” and “scanner” may be used interchangeably, as the description can relate to an image capture device that acquires image data in multiple modes, such as reflective color or monochrome images, contour images obtained from structured light, and image content acquired using OCT imaging techniques.

In the context of the present disclosure, the phrase “broadband light emitter” refers to a light source that emits a continuous spectrum output over a range of wavelengths at any given point of time. Low-coherence, broadband light sources can include, for example, super luminescent diodes, short-pulse lasers, and supercontinuum light sources.

According to an embodiment of the present disclosure, there is provided a programmable light source that can provide variable wavelength illumination. The programmable light source can be used as a swept-source for SS-OCT and other applications that benefit from a controllably changeable spectral pattern.

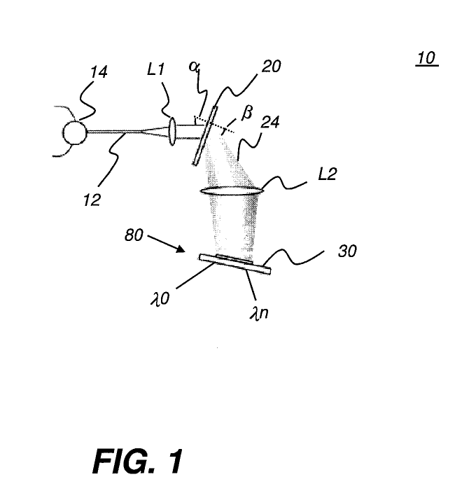

Referring to FIG. 1, there is shown a programmable filter 10 that is used for generating a desired pattern and sequence of wavelengths (λ0 . . . λn) from a low-coherence, broadband light source. Broadband light from a fiber laser or other source is directed, through a circulator 14 through an optical fiber or other waveguide 12 to a collimator lens L1 that directs the collimated light to a light dispersion optic 20, such as a diffraction grating. Light dispersion optic 20 forms a spectrally dispersed output beam 24, directed toward a focusing lens L2. Lens L2 focuses the dispersed light onto a spatial light modulator 80, such as a micro-mirror array 30. The micro-mirror array can be a linear array of reflective devices or a linear portion of a Digital Light Processor (DLP) from Texas Instruments, Dallas, Tex. One or more individual reflectors in array 30 is actuated to reflect light of corresponding wavelengths back through the optical path. This reflected light is the output of programmable filter 10 and can be used in applications such as optical coherence tomography (OCT) as described subsequently. Rapid actuation of each successive reflector in array 30 allows sampling of numerous small spectral portions of a spectrally dispersed output beam, such as that provided in FIG. 1. For example, where the spatial light modulator 80 is a micro-mirror array 30 that has 2048 micro-mirror elements in a single row, where the spectral range from one side of the array 30 to the other is 35 nm, each individual micro-mirror can reflect a wavelength band that is approximately 0.017 nm wide. One typical swept source sequence advances from lower to higher wavelengths by actuating a single spatial light modulator 80 pixel (reflective element) at a time, along the line formed by the spectrally dispersed output beam. Other swept source sequences are possible, as described subsequently.

The micro-mirror array 30 described herein and shown in FIGS. 1-3 and following is one type of possible spatial light modulator 80 that can be used as part of a programmable light source. The spatial light modulator 80 that is employed is a reflective device of some type, with discretely addressable elements that effectively provide the “pixels” of the device.

Programmable filter 10 resembles aspects of a spectrometer in its overall arrangement of components and in its light distribution. Incident broadband light is dispersed by light dispersion optic 20 in order to spatially separate the spectral components of the light. The micro-mirror array 30 or other type of spatial light modulator 80, as described in more detail subsequently, is disposed to reflect a selected wavelength band or bands of this light back through programmable filter 10 so that the selected wavelength band can be used elsewhere in the optical system, such as for use in an interferometry measurement device or for tuning a laser.

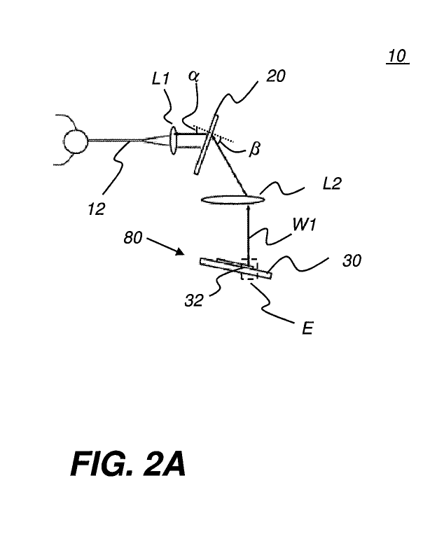

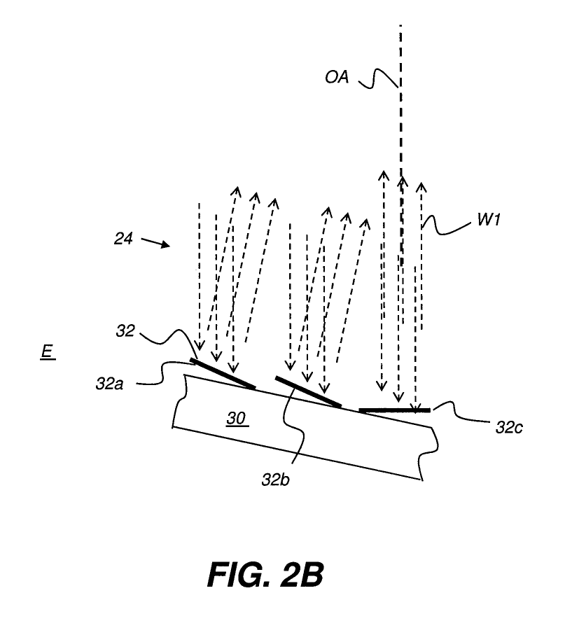

The simplified schematic of FIG. 2A and enlargement of FIG. 2B show how programmable filter 10 operates to provide light of a selected wavelength band W1. FIG. 2B, which schematically shows a greatly enlarged area E of micro-mirror array 30, shows the behavior of three mirrors 32a, 32b, and 32c with respect to incident light of beam 24. Each mirror 32 element of micro-mirror array 30 can have either of two states: deactuated, tilted at one angle, as shown at mirrors 32a and 32b; or actuated, tilted at an alternate angle as shown at mirror 32c. For DLP devices, the tilt angles for deactuated/actuated states of the micro-mirrors are +12 and −12 degrees from the substrate surface. Thus, in order to direct light back along optical axis OA through lens L2 and through the other components of programmable filter 10, micro-mirror array 30 is itself tilted at +12 degrees relative to the optical axis OA, as shown in FIG. 2B.

In the programmable filter 10 of FIG. 1, light dispersion optic 20 can be a diffraction grating of some type, including a holographic diffraction grating, for example. The grating dispersion equation is:

mλ=d(sin α+sin β) (eq. 1)

wherein:
λ is the optical wavelength;
d is the grating pitch;
α is the incident angle (see FIGS. 1, 2A), relative to a normal to the incident surface of optic 20;
β is the angle of diffracted light, relative to a normal to the exit surface of optic 20;
m is the diffraction order, generally m=1 with relation to embodiments of the present disclosure.

The FWHM (full-width half-maximum) bandwidth is determined by the spectral resolution of the grating δλ_{g} and wavelength range on a pixel or micro-mirror 32 of the DLP device δλ_{DLP}, which are given as:

δλ_{g}=λc d cos α/D (eq. 2)

and

δλ_{DLP}=dp cos β/f. (eq. 3)

wherein:
D is the 1/e² width of the incident Gaussian beam collimated by lens L1;
λc is the central wavelength;
d is the grating pitch;
p is the DLP pixel pitch, for each micro-mirror;
f is the focus length of focus lens L2.

The final FWHM bandwidth δλ is the maximum of (δλ_{g}, δλ_{DLP}). Bandwidth δλ, defines the finest tunable wavelength range. For a suitable configuration for OCT imaging, the following relationship holds:

δλ_{g}≤δλ_{DLP}.

In order to use the DLP to reflect the light back to the waveguide 12 fiber, the spectrally dispersed spectrum is focused on the DLP surface, aligned with the hinge axis of each micro-mirror 32. The DLP reference flat surface also tilts 12 degrees so that when a particular micro-mirror 32 is in an “on” state, the light is directly reflected back to the optical waveguide 12. When the micro-mirror is in an “on” state, the corresponding focused portion of the spectrum, with bandwidth corresponding to the spatial distribution of light incident on that micro-mirror, is reflected back to the waveguide 12 fiber along the same path of incident light, but traveling in the opposite direction. Circulator 14 in the fiber path guides the light of the selected spectrum to a third fiber as output. It can be readily appreciated that other types of spatial light modulator 80 may not require orientation at an oblique angle relative to the incident light beam, as was shown in the example of FIG. 2B.

The 1/e² Gaussian beam intensity diameter focused on a single DLP pixel is as follows:

w=4λ f/(π D cos β/cos α) (eq. 4)

Preferably, the following holds: w≤p. This sets the beam diameter w at less than the pixel pitch p. The maximum tuning range is determined by:

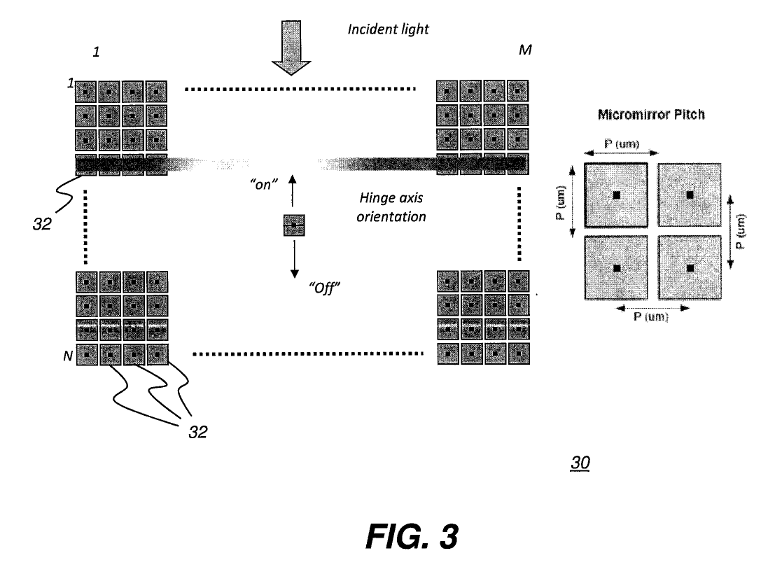

M×δλ_{DLP},

wherein M is the number of DLP micro-mirrors in the horizontal direction, as represented in FIG. 3. As FIG. 3 shows, the array of micro-mirrors for micro-mirror array 30 has M columns and N rows. Only a single row of the DLP micro-mirror array is needed for use with programmable filter 10; the other rows above and below this single row may or may not be used.

The wavelength in terms of DLP pixels (micro-mirrors) can be described by the following grating equation:

$${λ}_{i} = d ( sin α + sin ( {tan}^{- 1} [ \frac{p}{f} ( \frac{N}{2} - i - 1 ) ] + β ) ) \qquad ( eq . 5 )$$

Wherein i is an index for the DLP column, corresponding to the particular wavelength, in the range between 0 and (M−1).

From the above equation (5), the center wavelength corresponding to each mirror in the row can be determined.

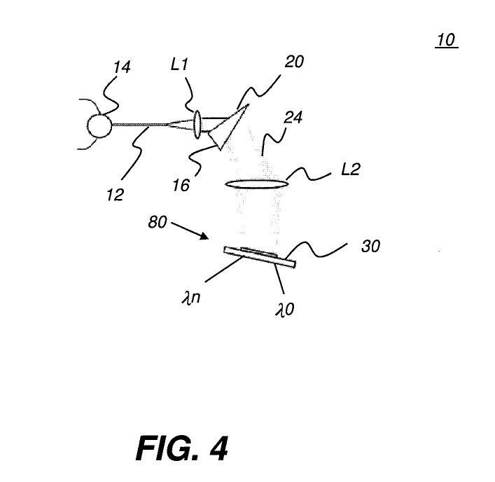

FIG. 4 shows programmable filter 10 in an alternate embodiment, with a prism 16 as light dispersion optic 20. The prism 16 disperses the light wavelengths (λn . . . λ0) in the opposite order from the grating shown in FIG. 1. Longer wavelengths (red) are dispersed at a higher angle, shorter wavelengths (blue) at lower angles.

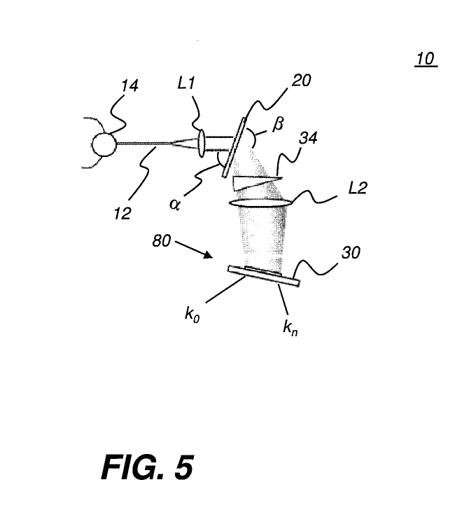

Conventional light dispersion optics distribute the dispersed light so that its constituent wavelengths have a linear distribution. That is, the wavelengths are evenly spaced apart along the line of dispersed light. However, for Fourier domain OCT processing, conversion of wavelength data to frequency data is needed. Wavelength data (λ in units of nm) must thus be converted to wave-number data (k=λ⁻¹), proportional to frequency. In conventional practice, an interpolation step is used to achieve this transformation, prior to Fourier transform calculations. The interpolation step requires processing resources and time. However, it would be most advantageous to be able to select wave-number k values directly from the programmable filter. The schematic diagram of FIG. 5 shows one method for optical conversion of wavelength (λ₀ . . . λ_{N}) data to wave-number (k₀ . . . k_{N}) data using an intermediate prism 34. Methods for specifying prism angles and materials parameters for wavelength-to-wavenumber conversion are given, for example, in an article by Hu and Rollins entitled “Fourier domain optical coherence tomography with a linear-in-wavenumber spectrometer” in OPTICS LETTERS, Dec. 15, 2007, vol. 32 no. 24, pp. 3525-3527.

Programmable filter 10 is capable of providing selected light wavelengths from a broadband light source in a sequence that is appropriately timed for functions such as OCT imaging using a tuned laser. Because it offers a programmable sequence, the programmable filter 10 can perform a forward spectral sweep from lower to higher wavelengths as well as a backward sweep in the opposite direction, from higher to lower wavelengths. A triangular sweep pattern, generation of a “comb” of wavelengths, or arbitrary wavelength pattern can also be provided.

For OCT imaging in particular, various programmable sweep paradigms can be useful to extract moving objects in imaging, to improve sensitivity fall-off over depth, etc. The OCT signal sensitivity decreases with increasing depth into the sample, with depth considered to extend in the z-axis direction. Employing a comb of discrete wavelengths, for example, can increase OCT sensitivity. This is described in an article by Bajraszewski et al. entitled “Improved spectral optical coherence tomography using optical frequency comb” in Optics Express, Vol. 16 No. 6, March 2008, pp. 4163-4176.

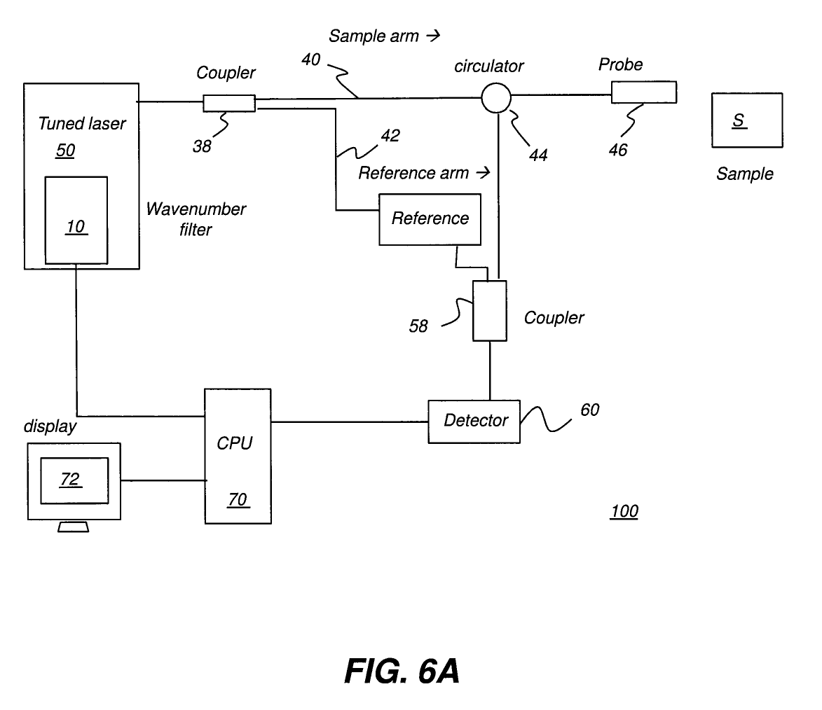

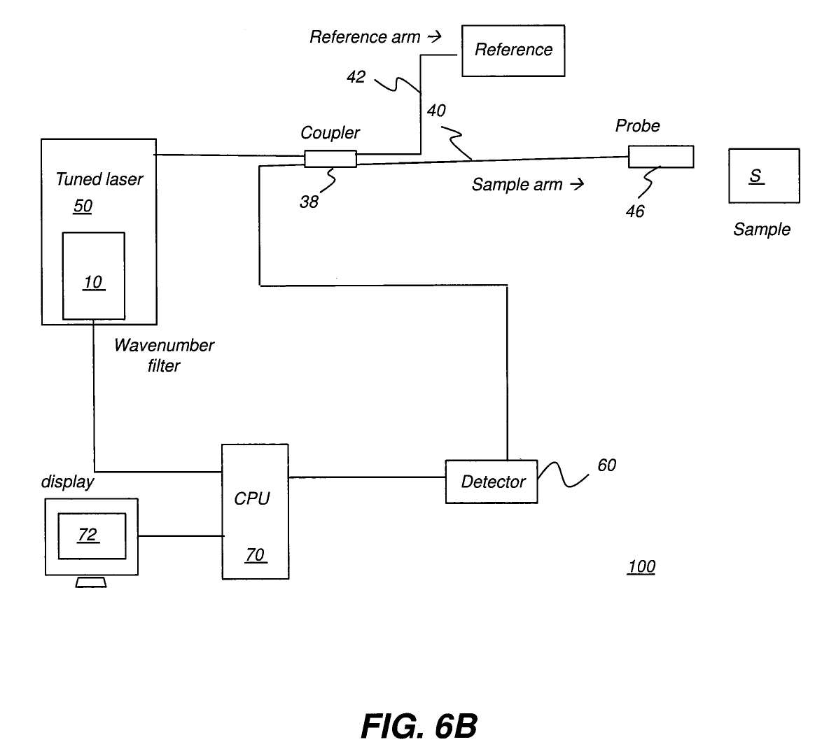

The simplified schematic diagrams of FIGS. 6A and 6B each show a swept-source OCT (SS-OCT) apparatus 100 using programmable filter 10 according to an embodiment of the present disclosure. In each case, programmable filter 10 is used as part of a tuned laser 50. For intraoral OCT, for example, laser 50 can be tunable over a range of frequencies (wave-numbers k) corresponding to wavelengths between about 400 and 1600 nm. According to an embodiment of the present disclosure, a tunable range of 35 nm bandwidth centered about 830 nm is used for intraoral OCT.

In the FIG. 6A embodiment, a Mach-Zehnder interferometer system for OCT scanning is shown. FIG. 6B shows components for a Michelson interferometer system. For these embodiments, programmable filter 10 provides part of the laser cavity to generate tuned laser 50 output. The variable laser 50 output goes through a coupler 38 and to a sample arm 40 and a reference arm 42. In FIG. 6A, the sample arm 40 signal goes through a circulator 44 and to a probe 46 for measurement of a sample S. The sampled signal is directed back through circulator 44 (FIG. 6A) and to a detector 60 through a coupler 58. In FIG. 6B, the signal goes directly to sample arm 40 and reference arm 42; the sampled signal is directed back through coupler 38 and to detector 60. The detector 60 may use a pair of balanced photodetectors configured to cancel common mode noise. A control logic processor (CPU) 70 is in signal communication with tuned laser 50 and its programmable filter 10 and with detector 60 and obtains and processes the output from detector 60. CPU 70 is also in signal communication with a display 72 for command entry and OCT results display.

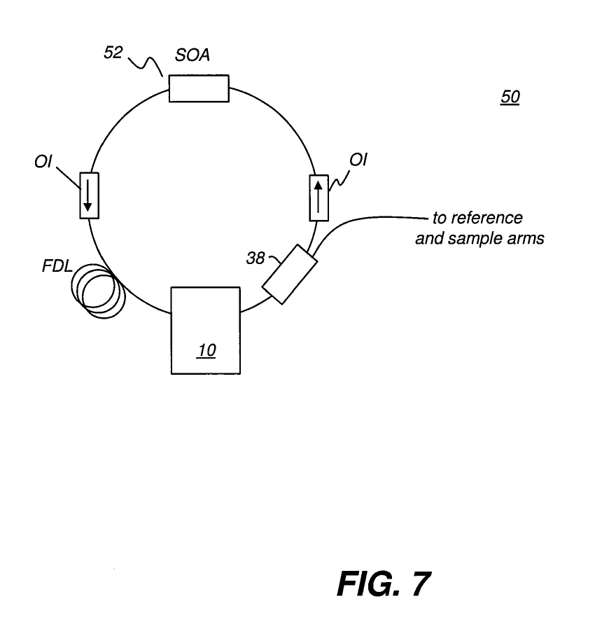

The schematic diagram of FIG. 7 shows components of tuned laser 50 according to an alternate embodiment of the present disclosure. Tuned laser 50 is configured as a fiber ring laser having a broadband gain medium such as a semiconductor optical amplifier (SOA) 52. Two optical isolators OI provide protection of the SOA from back-reflected light. A fiber delay line (FDL) determines the effective sweep rate of the laser. Filter 10 has an input fiber and output fiber, used to connect the fiber ring.

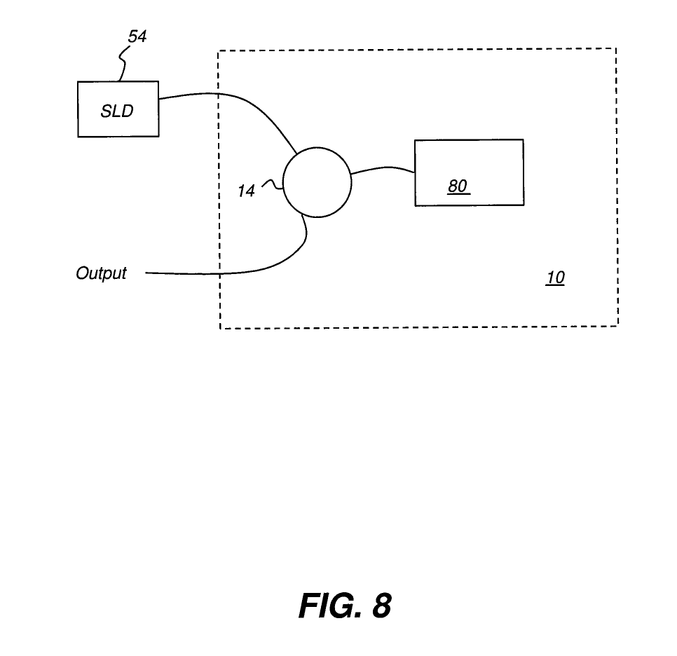

The schematic diagram of FIG. 8 shows the use of programmable filter 10 for selecting a wavelength band from a broadband light source 54, such as a super luminescent diode (SLD). Here, spatial light modulator 80 reflects a component of the broadband light through circulator 14. Circulator 14 is used to direct light to and from the programmable filter 10 along separate optical paths.

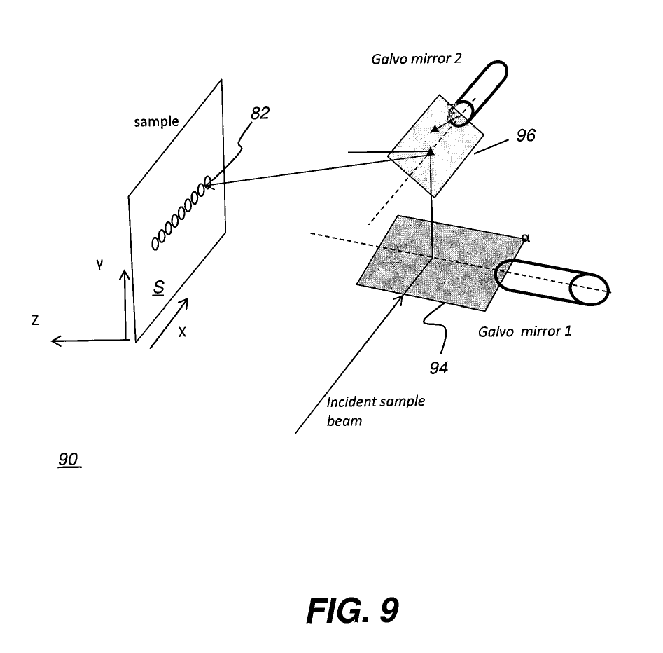

As shown in the schematic diagram of FIG. 9, galvo mirrors 94 and 96 cooperate to provide the raster scanning needed for OCT imaging. In the arrangement that is shown, galvo mirror 1 (94) scans the wavelengths of light to each point 82 along the sample to generate data along a row, which provides the B-scan, described in more detail subsequently. Galvo mirror 2 (96) progressively moves the row position to provide 2-D raster scanning to additional rows. At each point 82, the full spectrum of light provided using programmable filter 10, pixel by pixel of the spatial light modulator 80 (FIGS. 1, 4, 5), is rapidly generated in a single sweep and the resulting signal measured at detector 60 (FIGS. 6A, 6B).

### Scanning Sequence

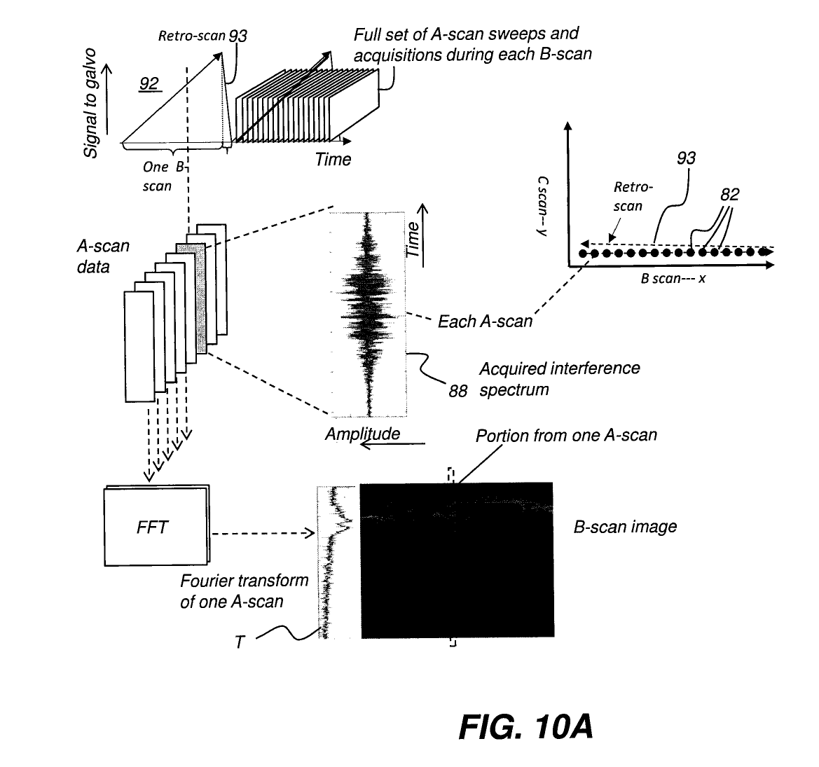

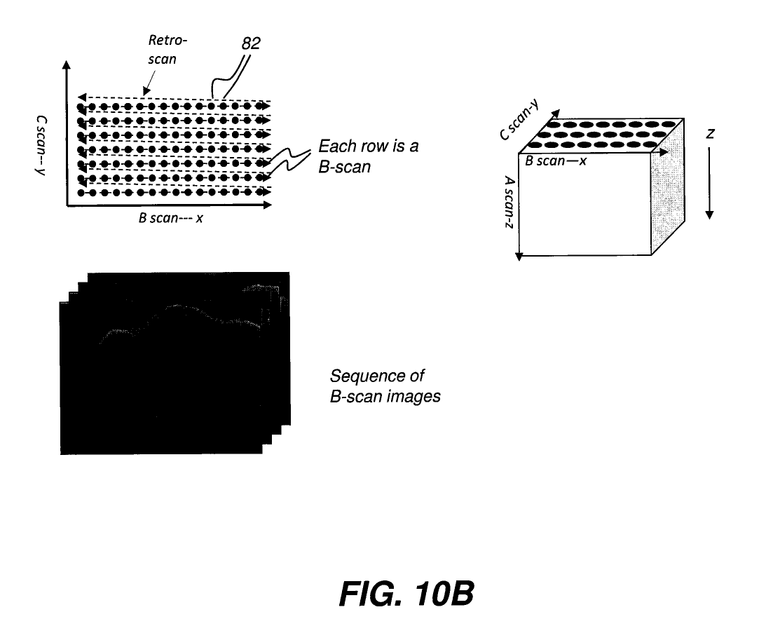

The schematic diagrams of FIGS. 10A and 10B show a scan sequence that can be used for forming tomographic images using the OCT apparatus of the present disclosure. The sequence shown in FIG. 10A shows how a single B-scan image is generated. Scanner 90 (FIG. 9) scans the selected light sequence over sample S, point by point. A periodic drive signal 92 as shown in FIG. 10A is used to drive the scanner galvo mirrors to control a lateral scan or B-scan that extends across each row of the sample, shown as discrete points 82 extending in the horizontal direction in FIGS. 10A and 10B. At each of a plurality of points 82 along a line or row of the B-scan, an A-scan or depth scan, acquiring data in the z-axis direction, is generated using successive portions of the selected wavelength band. FIG. 10A shows drive signal 92 for generating a straightforward ascending sequence, with corresponding micro-mirror actuations, or other spatial light modulator pixel-by-pixel actuation, through the wavelength band. The retro-scan signal 93, part of drive signal 92, simply restores the scan mirror back to its starting position for the next line; no data is obtained during retro-scan signal 93.

It should be noted that the B-scan drive signal 92 drives the galvo mirror 94 for scanner 90 as shown in FIG. 9. At each incremental position, point 82 along the row of the B-scan, an A-scan is obtained. To acquire the A-scan data, tuned laser 50 or other programmable light source sweeps through the spectral sequence that is controlled by programmable filter 10 (FIGS. 1, 2A, 4, 5). Thus, in an embodiment in which programmable filter 10 causes the light source to sweep through a 30 nm range of wavelengths, this sequence is carried out at each point 82 along the B-scan path. As FIG. 10A shows, the set of A-scan acquisitions executes at each point 82, that is, at each position of the scanning galvo mirror 94. By way of example, where a DLP micro-mirror device is used as spatial light modulator 80, there can be 2048 measurements for generating the A-scan at each position 82.

FIG. 10A schematically shows the information acquired during each A-scan. An interference signal 88, shown with DC signal content removed, is acquired over the time interval for each point 82, wherein the signal is a function of the time interval required for the sweep, with the signal that is acquired indicative of the spectral interference fringes generated by combining the light from reference and feedback arms of the interferometer (FIGS. 6A, 6B). The Fourier transform generates a transform T for each A-scan. One transform signal corresponding to an A-scan is shown by way of example in FIG. 10A.

From the above description, it can be appreciated that a significant amount of data is acquired over a single B-scan sequence. In order to process this data efficiently, a Fast-Fourier Transform (FFT) is used, transforming the time-based signal data to corresponding frequency-based data from which image content can more readily be generated.

In Fourier domain OCT, the A scan corresponds to one line of spectrum acquisition which generates a line of depth (z-axis) resolved OCT signal. The B scan data generates a 2D OCT image along the corresponding scanned line.

Raster scanning is used to obtain multiple B-scan data by incrementing the scanner acquisition in the C-scan direction. This is represented schematically in FIG. 10B, which shows how 3-D volume information is generated using the A-, B-, and C-scan data.

As noted previously, the wavelength or frequency sweep sequence that is used at each A-scan point 82 can be modified from the ascending or descending wavelength sequence that is typically used. Arbitrary wavelength sequencing can alternately be used. In the case of arbitrary wave selection, which may be useful for some particular implementations of OCT, only a portion of the available wavelengths are provided as a result of each sweep. In arbitrary wavelength sequencing, each wavelength can be randomly selected to be used in the OCT system during a single sweep.

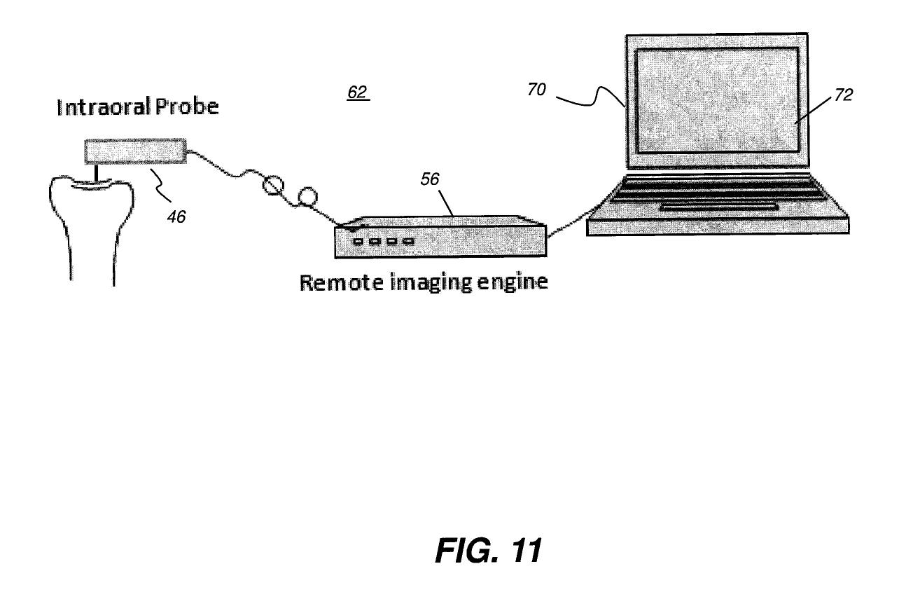

The schematic diagram of FIG. 11 shows probe 46 and support components for forming an intraoral OCT imaging system 62. An imaging engine 56 includes the light source, fiber coupler, reference arm, and OCT detector components described with reference to FIGS. 6A-7. Probe 46, in one embodiment, includes the raster scanner or sample arm, but may optionally also contain other elements not provided by imaging engine 56. CPU 70 includes control logic and display 72.

The preceding description gives detailed description of OCT imaging system 62 using a DLP micro-mirror array 30 as one useful type of spatial light modulator that can be used for selecting a wavelength band from programmable filter 10. However, it should be noted that other types of spatial light modulator 80 could be used to reflect light of a selected wavelength band. A reflective liquid crystal device could alternately be used in place of DLP micro-mirror array 30, for example. Other types of MEMS (micro-electromechanical system devices) micro-mirror array that are not DLP devices could alternately be used.

Consistent with an embodiment of the present invention, a computer program utilizes stored instructions that perform on image data that is accessed from an electronic memory. As can be appreciated by those skilled in the image processing arts, a computer program for operating the imaging system in an embodiment of the present disclosure can be utilized by a suitable, general-purpose computer system operating as CPU 70 as described herein, such as a personal computer or workstation. However, many other types of computer systems can be used to execute the computer program of the present invention, including an arrangement of networked processors, for example. The computer program for performing the method of the present invention may be stored in a computer readable storage medium. This medium may comprise, for example; magnetic storage media such as a magnetic disk such as a hard drive or removable device or magnetic tape; optical storage media such as an optical disc, optical tape, or machine readable optical encoding; solid state electronic storage devices such as random access memory (RAM), or read only memory (ROM); or any other physical device or medium employed to store a computer program. The computer program for performing the method of the present disclosure may also be stored on computer readable storage medium that is connected to the image processor by way of the internet or other network or communication medium. Those skilled in the art will further readily recognize that the equivalent of such a computer program product may also be constructed in hardware.

It should be noted that the term “memory”, equivalent to “computer-accessible memory” in the context of the present disclosure, can refer to any type of temporary or more enduring data storage workspace used for storing and operating upon image data and accessible to a computer system, including a database, for example. The memory could be non-volatile, using, for example, a long-term storage medium such as magnetic or optical storage. Alternately, the memory could be of a more volatile nature, using an electronic circuit, such as random-access memory (RAM) that is used as a temporary buffer or workspace by a microprocessor or other control logic processor device. Display data, for example, is typically stored in a temporary storage buffer that is directly associated with a display device and is periodically refreshed as needed in order to provide displayed data. This temporary storage buffer is also considered to be a type of memory, as the term is used in the present disclosure. Memory is also used as the data workspace for executing and storing intermediate and final results of calculations and other processing. Computer-accessible memory can be volatile, non-volatile, or a hybrid combination of volatile and non-volatile types.

It will be understood that the computer program product of the present disclosure may make use of various image manipulation algorithms and processes that are well known. It will be further understood that the computer program product embodiment of the present disclosure may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with the computer program product of the present disclosure, are not specifically shown or described herein and may be selected from such algorithms, systems, hardware, components and elements known in the art.

The invention has been described in detail, and may have been described with particular reference to a suitable or presently preferred embodiment, but it will be understood that variations and modifications can be effected within the spirit and scope of the invention. In addition, while a particular feature of the invention can have been disclosed with respect to one of several implementations, such feature can be combined with one or more other features of the other implementations as can be desired and advantageous for any given or particular function. The presently disclosed embodiments are therefore considered in all respects to be illustrative and not restrictive. Embodiments according to the application can include various features described herein (individually or in combination). The scope of the invention is indicated by the appended claims, and all changes that come within the meaning and range of equivalents thereof are intended to be embraced therein.

1. A programmable light source comprising:
a) a broadband light emitter disposed to direct light to an optical filter;
b) the optical filter having:
(i) a collimator lens in the path of the directed light from the emitter;
(ii) a dispersion optic in the path of incident light from the collimator lens and angularly disposed to form a spectrally dispersed output beam from the incident beam;
(iii) a focusing lens in the path of the spectrally dispersed output beam;
(iv) a spatial light modulator in the focal region of the focusing lens, the spatial light modulator disposed to reflect sequential spectral portions of the spectrally dispersed output beam back toward the focusing lens.

2. The light source according to claim 1 wherein the spatial light modulator comprises an array of independently tiltable reflective surfaces in the focal plane of the focusing lens, wherein each reflective surface in the array is responsive to a control signal to orient to a first tilt state at a first angle that redirects incident light back toward the focusing lens or to a second tilt state at a second angle, wherein each reflective surface in the array is in the path of incident light of a corresponding wavelength range from the focused, spectrally dispersed output beam.

3. The light source according to claim 1 wherein the spatial light modulator is a digital micro-mirror array.

4. The light source according to claim 1 wherein the spatial light modulator is disposed to generate an ascending sweep through successively increasing wavelengths of the spectrally dispersed output beam.

5. The light source according to claim 1 wherein the spatial light modulator is disposed to generate a descending sweep through successively decreasing wavelengths of the spectrally dispersed output beam.

6. The light source according to claim 1 wherein the spatial light modulator is disposed to generate an arbitrary sequence of wavelengths of the spectrally dispersed output beam.

7. The light source according to claim 1 wherein the spatial light modulator is disposed to generate a series of discrete wavelengths of the spectrally dispersed output beam.

8. The light source according to claim 1 wherein the light dispersion optic is a diffraction grating.

9. The light source according to claim 8 further comprising a prism in the path of the spectrally dispersed output beam from the diffraction grating.

10. The light source according to claim 1 wherein the light dispersion optic is a prism.

11. The light source according to claim 1 further comprising an optical circulator in the path of light from the broadband light emitter.

12. An optical coherence tomography imaging apparatus comprising:
an interferometer having:
a) a programmable light source having:
a broadband light emitter disposed to direct light to an optical filter;
the optical filter having:
(i) a collimator lens in the path of the directed light from the emitter;
(ii) a dispersion optic in the path of incident light from the collimator lens and angularly disposed to form a spectrally dispersed output beam from the incident beam;
(iii) a focusing lens in the path of the spectrally dispersed output beam;
(iv) a spatial light modulator in the focal region of the focusing lens, the spatial light modulator disposed to reflect sequential spectral portions of the spectrally dispersed output beam back toward the focusing lens;
b) a reference arm in the path of a reference portion of an output beam from the programmable light source;
c) a sample arm comprising a probe having a scanning apparatus actuable to scan a sample portion of the output beam of the programmable light source toward a sample in a raster scan pattern and to obtain reflected light from the sample;
d) a photodetector disposed to generate an output signal according to optical interference between the sensed reflected light and the reference portion of the output beam;
a processor that follows programmed instructions to receive the generated output signal, execute Fourier transform calculations on the received signal, and generate tomographic image data according to the calculations; and
a display that is in signal communication with the processor and is energizable to display the generated tomographic image data.

13. The optical coherence tomography imaging apparatus according to claim 12 wherein the probe is configured for intraoral use.

14. The optical coherence tomography imaging apparatus according to claim 12 wherein the broadband light emitter is a super luminescent diode.

15. A fiber ring laser comprising
(a) a broadband gain medium disposed to direct light to an optical filter;
(b) the optical filter having:
(i) a collimator lens in the path of emitter light from the optical circulator and disposed to direct an incident beam to a light dispersion optic;
(ii) the light dispersion optic disposed to form a spectrally dispersed output beam from the incident beam;
(iii) a focusing lens in the path of the spectrally dispersed output beam;
(iv) a spatial light modulator in the focal region of the focusing lens, the spatial light modulator disposed to reflect sequential spectral portions of the spectrally dispersed output beam back toward the focusing lens; and
(c) a fiber delay line that sets the sweep rate of the fiber ring laser.

16. An optical coherence tomography imaging apparatus comprising:
an interferometer having:
(a) a fiber ring laser comprising:
a broadband gain medium disposed to direct light to an optical filter;
the optical filter having:
(i) a collimator lens in the path of emitter light from the optical circulator and disposed to direct an incident beam to a light dispersion optic;
(ii) the light dispersion optic disposed to form a spectrally dispersed output beam from the incident beam;
(iii) a focusing lens in the path of the spectrally dispersed output beam;
(iv) a spatial light modulator in the focal region of the focusing lens, the spatial light modulator disposed to reflect sequential spectral portions of the spectrally dispersed output beam back toward the focusing lens; and
a fiber delay line that sets the sweep rate of the fiber ring laser.
b) a reference arm in the path of a reference portion of an output beam from the programmable light source;
c) a sample arm comprising a probe having a scanning apparatus actuable to scan a sample portion of the output beam of the programmable light source toward a sample in a raster scan pattern and to obtain reflected light from the sample;
d) a photodetector disposed to generate an output signal according to optical interference between the sensed reflected light and the reference portion of the output beam;
a processor that executes programmed instructions to receive the generated output signal, execute Fourier transform calculations on the received signal, and generate tomographic image data according to the calculations; and
a display in signal communication with the processor and energizable to display the generated tomographic image data.

17. The optical coherence tomography imaging apparatus according to claim 16 wherein the probe is configured for intraoral use.

18. The optical coherence tomography imaging apparatus according to claim 16 wherein the spatial light modulator has an array of micro-mirrors.

19. The optical coherence tomography imaging apparatus according to claim 16 wherein the broadband gain medium is a semiconductor optical amplifier.

20. A programmable light source comprising:
(a) a broadband light emitter disposed to direct light to an optical circulator; and
(b) an optical filter disposed to obtain light of one or more selected wavelength ranges from the broadband emitter through the optical circulator, the optical filter having:
(i) a collimator lens in the path of emitter light from the optical circulator and disposed to direct an incident beam to a light dispersion optic;
(ii) the light dispersion optic disposed to form a spectrally dispersed output beam from the incident beam;
(iii) a focusing lens in the path of the spectrally dispersed output beam;
(iv) an array of independently tiltable reflective surfaces in the focal plane of the focusing lens, wherein each reflective surface in the array is responsive to a control signal to orient to a first tilt state at a first angle that redirects incident light back toward the focusing lens or to a second tilt state at a second angle, wherein each reflective surface in the array is in the path of incident light of a corresponding wavelength range from the focused, spectrally dispersed output beam.
